(19) United States
(12) Patent Application Publication
(10) Pub. No.: US 2002/0142017 A1
(43) Pub. Date: Oct. 3, 2002

(54) Suspension of nanospheres of lipophilic active principle stabilized with water-dispersible polymers

(76) Inventor: Jean-Thierry Simonnet, Paris (FR)

Correspondence Address: NIXON & VANDERHYE P.C., 1100 North Glebe Road, 8th Floor, Arlington, VA, 22201, US

(21) Appl. No.: 10/060,280

(22) Filed: Feb. 1, 2002

(30) Foreign Application Priority Data
Feb. 2, 2001 (FR) 01 01438

(51) Int. Cl. A61K 31/7048; A61K 31/56; A61K 31/19

(52) U.S. Cl. 424/401; 514/27; 514/171; 514/557; 514/733

(57) ABSTRACT

The present invention relates to a stable colloidal suspension consisting essentially:

of a continuous aqueous phase,

of nanospheres of lipophilic active principle with a mean particle size ranging from 0.01 to 1 μm,

of at least one surfactant, and

of colloidal particles, with a mean size ranging from 10 to 500 nm, of at least one water-dispersible polymer,

and also to topical-application compositions containing such a colloidal suspension.

The invention also relates to the use of water-dispersible polymers in the form of colloidal particles with a mean size ranging from 10 to 500 nm, to stabilize aqueous suspensions of nanospheres of lipophilic active principle, with a mean particle size ranging from 0.01 nm to 1 μm, against the recrystallization of the active principle.

The present invention relates to aqueous suspensions of nanospheres of lipophilic active principles, stabilized with particles of water-dispersible polymers, and also to topical-application compositions containing them.

There are many cosmetic active principles which, on account of their highly lipophilic nature, are insoluble in the majority of cosmetically acceptable solvents and are consequently difficult to incorporate into cosmetic compositions.

One advantageous approach for solving this insolubility problem consists in forming with these lipophilic molecules solid particles that are very small (smaller than one micron), known as nanoparticles, which may then be used in the form of colloidal aqueous suspensions.

Among nanoparticles, nanocapsules and nanospheres may be distinguished.

Nanocapsules are particles of core-envelope structure. The inner phase containing the active principle in dissolved, dispersed or pure form, is encapsulated in a solid continuous envelope that is insoluble in the medium, and that is generally of polymeric or waxy nature.

Nanospheres are solid spheres consisting of pure solid active principle or of active principle incorporated into a waxy or polymeric matrix.

The nanospheres under consideration in the present invention are those consisting of pure solid active principle. The active principle therein is in amorphous form. However, beyond a certain critical concentration of active principle, this amorphous form may evolve by recrystallization of the active agent under consideration.

In general, the instability of aqueous suspensions of nanospheres of lipophilic active principle is reflected by the self-aggregation of the particles and/or the uncontrolled recrystallization of the active principle, especially under the effect of what is known as “Ostwald maturation” as described, for example, in the article by Kabalnof et al., J. Colloid and Interface Sci., 118 (1987), pages 590 to 597. This evolution is ultimately reflected by sedimentation of the particles or even by a setting of these particles to a solid, naturally leading to a reduced bioavailability of the active principle if it is dispersed in a cosmetic or dermatological support.

Now, the fact that it is impossible to prepare stable aqueous suspensions with large contents of active principle represents a considerable drawback for cosmetics formulators. Specifically, the addition of the active principle in the form of dilute suspensions involves the undesirable introduction of a large fraction of aqueous phase that is liable to modify the physicochemical properties of the cosmetic composition.

There is thus a need to have available stable aqueous suspensions containing high concentrations of nanospheres of lipophilic active principle.

The Applicant has thus sought to formulate biologically active lipophilic active principles, in the form of stable aqueous suspensions of nanospheres smaller than one micron in size and with a high concentration of active principle.

The Applicant has discovered, surprisingly, that it is possible to stabilize aqueous suspensions of nanoshperes of lipophilic active principles in amorphous form by adding thereto colloidal particles of at least one water-dispersible polymer. The addition of a water-dispersible polymer, usually in the form of colloidal particles in aqueous suspension, to an aqueous suspension of nanospheres of active principle makes it possible to significantly increase the critical concentration beyond which the nanospheres of active principle in suspension show a tendency towards uncontrolled crystallization.

One subject of the present invention is consequently a stable colloidal suspension consisting essentially:

of a continuous aqueous phase,

of nanospheres of lipophilic active principle with a mean particle size ranging from 0.01 to 1 μm (i.e. 10 nm to 1 μm),

of at least one surfactant, and

of colloidal particles, with a mean size ranging from 10 to 500 nm, of at least one water-dispersible polymer.

The water-dispersible polymer is preferably present in an amount that is sufficient to stabilize the nanospheres against recrystallization of the active principle.

A subject of the invention is also a topical-application composition containing, in a physiologically acceptable medium, such a stable colloidal suspension.

The expression “topical application” means herein an external application to keratin materials, which are especially the skin, the scalp, the eyelashes, the eyebrows, the nails and mucous membranes. The composition may be in particular a cosmetic or dermatological composition.

The expression “physiologically acceptable medium” means a medium that is compatible with skin tissues and able to be applied to the entire human body, and especially to the skin, the scalp, the eyelashes, the eyebrows, the nails and mucous membranes.

A subject of the invention is also the use of water-dispersible polymers in the form of colloidal particles with a mean size ranging from 10 to 500 nm, to stabilize aqueous suspensions of nanospheres of lipophilic active principle, having a mean particle size ranging from 0.01 to 1 μm, against recrystallization of the active principle.

The lipophilic active principles used in the aqueous suspensions of the present invention have a solubility in water at room temperature (25° C.) of less than 0.01% and a solubility of less than 7.5% in Guerbet alcohols such as octyldodecanol, and in glycols such as, for example, glycerol, polyethylene glycols or isoprene glycol.

They are, moreover, solid at room temperature and preferably have a melting point of greater than 100° C.

Examples of lipophilic active principles placed in the form of nanospheres in aqueous suspensions that may be mentioned according to the present invention are those belonging to the following families:

(1) Sterols of plant or animal origin such as cholesterol, ergosterol, campesterol, stigmasterol, brassicasterol and sitosterol, partially hydrogenated derivatives of these sterols (=stanols), and also esters thereof.

(2) Dehydroepiandrosterone (DHEA) and chemical and biological precursors and derivatives thereof; dehydroepiandrosterone is a natural steroid, produced essentially by the adrenocortical glands, corresponding to the formula

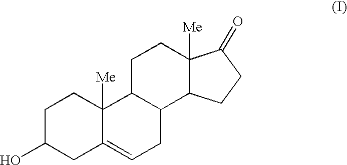

It is known for its anti-ageing properties associated with its capacity to promote epidermal keratinization (JP-07 196 467) and to combat osteoporosis (U.S. Pat. No. 5,824,671), or in the treatment of dry skin, on account of its ability to increase the endogenous production and secretion of sebum and to reinforce the skin's barrier effect (U.S. Pat. No. 4,496,556). It has also been proposed to use DHEA sulphate against alopecia (JP-60 142 908) and to treat various signs of ageing such as wrinkles, loss of radiance of the skin and slackening of the skin (EP-0 723 775).

The DHEA that may be used according to the invention is available, for example, from the companies Sigma and Akzo Nobel.

The expression “DHEA precursor” means the immediate biological precursors thereof and also the chemical precursors thereof. Examples of biological precursors are cholesterol, pregnenolone, 17α-hydroxypregnenolone, 5-androstenediol, 17α-hydroxypregnenolone sulphate and 5-androstenediol sulphate. Examples of chemical precursors are sapogenins such as diosgenin (spirost-5-ene-3-beta-ol), hecogenin, smilagenin and sarsapogenin, and also natural extracts containing them, in particular fenugreek and extracts of Dioscorea plants such as wild yam root.

The expression “DHEA derivatives” means both the metabolic derivatives thereof and the chemical derivatives thereof. Metabolic derivatives which may be mentioned in particular include 7α-hydroxy-DHEA, 7-keto-DHEA, 5-androstene-30β,17β-diol (or adiol), 5-androstene-30β,170β-diol sulphate and 4-androstene-3,17-dione, although this list is not limiting. Chemical derivatives that may be mentioned in particular include salts, in particular water-soluble salts such as DHEA sulphate, esters such as the hydroxycarboxylic acid esters of DHEA described in U.S. Pat. No. 5,736,537 or other esters such as DHEA salicylate, acetate, valerate and enanthate;

(3) Pentacyclic triterpene acids such as ursolic acid and oleanolic acid. They are present in plants such as rosemary. They are frequently used in pharmaceutical compositions for their numerous therapeutic properties, and especially for their anti-inflammatory, hepato-protective, diuretic, analgesic and antimicrobial properties, their inhibitory properties on certain enzymatic activities, and their antitumour properties. In the cosmetic field, ursolic acid is described, for example, as a constituent of an antiperspirant composition (FR A 2 541 895) and as an inhibitor of the activity of tyrosinase, a key enzyme in melanin synthesis (JP-58/57307).

(4) Hydroxystilbenes, which are compounds corresponding to the general formula:

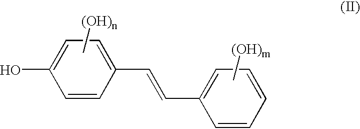

in which n is an integer between 1 and 4 inclusive and m is an integer between 1 and 5 inclusive. This formula includes the cis and trans compounds. According to the present invention, the term “hydroxystilbene” also covers the hydroxyalkyl derivatives of the compounds of formula (II). Hydroxystilbenes are compounds that are found in the natural state in plants of the spermatophyte class and in particular in vine. In the cosmetic field, hydroxystilbenes are used, inter alia, as depigmenting agents (JP-87-192 040) or anti-ageing agents (FR-2 777 186). Among the hydroxystilbenes that may be mentioned are mono-, di-, tri-, tetra-, penta-, hexa-, hepta-, octa- and nonahydroxystilbenes, or hydroxyalkyl derivatives thereof. According to the invention, the hydroxystilbenes may be used alone or as mixtures of any nature and may be of natural or synthetic origin. The hydroxystilbenes that may be used according to the invention are chosen from:

4-hydroxystilbene,

2′,4′-dihydroxystilbene,

3′,4′-dihydroxystilbene,

4,4′-dihydroxystilbene,

2′,4′,4-trihydroxystilbene,

3′,4′,4-trihydroxystilbene,

2,4,4′-trihydroxystilbene,

3,4,4′-trihydroxystilbene,

3,5,4′-trihydroxystilbene,

2′,3,4-trihydroxystilbene,

2,3′,4-trihydroxystilbene,

2′,2,4′-trihydroxystilbene,

2,4,4′,5′-tetrahydroxystilbene,

2′,3,4′,5-tetrahydroxystilbene,

2,2′,4,4′-tetrahydroxystilbene,

3,3′,4′,5-tetrahydroxystilbene,

2,3′,4,4′-tetrahydroxystilbene,

3,3′,4,4′-tetrahydroxystilbene,

3,3′,4′,5,5′-pentahydroxystilbene,

2,2′,4,4′,6-pentahydroxystilbene,

2,3′,4,4′,6-pentahydroxystilbene,

2,2′,4,4′,6,6′-hexahydroxystilbene.

Among these compounds, resveratrol (3,5,4′-trihydroxystilbene) is particularly preferred since it is naturally present in the skin of grape seeds and in wine. In this regard, the review by Soleas and collaborators (Clinical Biochemistry, vol. 30, No. 2, pages 91-113, 1997), which perfectly summarizes the state of knowledge regarding this compound and hydroxystilbenes, may be consulted.

(5) Isoflavonoids, a sub-class of flavonoids, which are formed from a 3-phenylchroman skeleton that is oxidized to a greater or lesser extent and that may bear various substituents. The term “isoflavonoid” covers several classes of compounds, among which mention may be made of isoflavones, isoflavanones, rotenoids, pterocarpans, isoflavans, isoflavan-3-enes, 3-arylcoumarins, 3-aryl-4-hydroxycoumarins, coumestanes, coumaronochromones or 2-arylbenzo-furans. A full review of isoflavonoids, their sources and methods of analysis has been published in “The Flavonoids”, Harbone editor (1988), chapter 5 entitled “Isoflavonoids” by P. M. Dewick, pages 125-157. The isoflavonoids used according to the invention have a solubility in water at room temperature (25° C.) of less than 0.01% and may be of natural origin, that is to say extracts of an element of natural origin, usually a plant, or may have been obtained by chemical synthesis. Isoflavonoids of natural origin are preferred. An example of an isoflavonoid of natural origin that may be mentioned is genistin. A preferred sub-class of isoflavonoids is that of isoflavones, covering both the aglycone forms (daidzein, genistein and glycitein) and the glycosyl forms (daidzin, genistin, glycitin). Processes for preparing isoflavones are described in particular in patents and patent applications WO 95/10530, WO 95/10512, U.S. Pat. Nos. 5,679,806, 5,554,519, EP 812 837 and WO 97/26269.

Isoflavones are known in particular as antioxidants, for their free-radical-scavenging and depigmenting properties, and also for their capacity to inhibit the activity of sebaceous glands (DE-44 32 947). They have also been described as agents capable of preventing signs of ageing of the skin (JP 1-96106).

(6) Aminophenol derivatives of formula

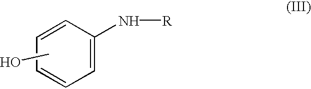

in which R is a radical corresponding to one of the formulae (i), (ii) and (iii) below

—CO—NR₁R₂ (i)

—CO—O—R₃ (ii)

—SO₂—R₃ (iii)

in which

R₁ represents a hydrogen atom or an optionally hydroxylated, saturated or unsaturated, linear or branched C₁₋₆ alkyl radical,

R₂ represents a hydrogen atom or a radical chosen from optionally hydroxylated, saturated or unsaturated, linear, cyclic or branched C₁₂ to C₃₀ alkyl radicals, and

R₃ represents a radical chosen from saturated or unsaturated, linear, branched or cyclic C₁₂ to C₃₀ alkyl radicals, including fused polycyclic radicals, that are optionally hydroxylated.

Examples of active principles that are preferred according to the present invention which may be mentioned include dehydroepiandrosterone (DHEA), DHEA sulphate, 7α-hydroxy-DHEA, 7-keto-DHEA, prednisolone, prednisone, progesterone, pregnenolone, testosterone, diosgenin, hecogenin, ursolic acid, oleanolic acid, resveratrol (=3,5,4′-trihydroxystilbene) and N-cholesteryloxycarbonyl-4-aminophenol, and isoflavonoids whose solubility in water at room temperature (25° C.) is less than 0.01%.

The colloidal suspensions of the present invention contain, in addition to the aqueous phase and the nanospheres of lipophilic active principle, one or more surfactants chosen from nonionic, anionic, cationic and zwitterionic surfactants, or mixtures thereof. These surfactants are introduced during the process for preparing the aqueous suspensions of nanospheres. Depending on their hydrophilic or lipophilic nature, the surfactant(s) is(are) then dissolved in the aqueous and/or organic phase. In the final aqueous suspension of nanospheres, the surfactants may be found dissolved in the aqueous continuous phase, adsorbed onto the surface of the nanospheres or incorporated in the nanospheres of lipophilic active principle.

The overall concentration of the surfactant(s) used preferably ranges from 0.02% to 25% and better still from 0.05% to 10%, relative to the total weight of the final aqueous suspension of nanospheres.

Examples which may be mentioned of preferred surfactants that may be used in the aqueous suspensions of nanospheres according to the present invention include those forming part of the following families:

(a) natural or synthetic, hydrogenated or non-hydrogenated phospholipids, optionally enriched with phosphatidylcholine. Examples that may be mentioned include soybean lecithin enriched with 45% by weight of phosphatidylcholine (sold under the name Emulmetik® 900 by the company Lucas Meyer) or a hydrogenated soybean lecithin (sold under the name Lécinol® S10 by the company Nikkol or under the name Emulmetik® 950 by the company Lucas Meyer);

(b) polyethoxylated sterols such as cholesterol and phytosterol polyethoxylated with 5 to 100 ethylene oxide (EO) units;

(c) surfactants chosen from fatty esters of glycerol, fatty esters of sorbitan, polyethoxylated fatty esters of sorbitan, polyethoxylated fatty alcohols and polyethoxylated fatty acids, the fatty chains of these molecules being saturated, linear or branched C₁₂₋₃₀ chains; examples that may be mentioned include behenyl alcohol polyethoxylated with 30 EO, stearic acid polyethoxylated with 40 EO or sorbitan laurate polyethoxylated with 20 EO;

(d) poly(vinyl alcohol), polyvinylpyrrolidone and copolymers thereof;

(e) polyethoxylated, and optionally poly-propoxylated, polysiloxanes (CTFA name: dimethicone copolyols), such as, for example, those described in patents U.S. Pat. Nos. 5,364,633 and 5,411,744. These silicone surfactants correspond in particular to the formula

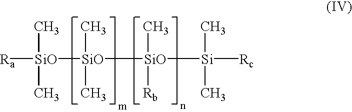

in which

Rₐ, R_{b} and R_{c} each independently represent a C₁₋₆ alkyl radical or a radical —(CH₂)ₓ—(O—CH₂—CH₂)_{y}—(OCH₂CH₂CH₂)_{z}OR_{d} in which R_{d} represents a hydrogen atom or an alkyl or acyl radical, at least one of the radicals Rₐ, R_{b} and R_{c} not being a C₁₋₆ alkyl radical,

m is an integer ranging from 0 to 200,

n is an integer ranging from 0 to 50, the sum m+n being other than zero,

x is an integer ranging from 1 to 6,

y is an integer ranging from 1 to 30, and

z is an integer ranging from 0 to 5.

According to one preferred embodiment of the invention, the alkyl radicals Rₐ, R_{b} R_{c} or R_{d} represent a methyl group, x is an integer ranging from 2 to 6 and y is an integer ranging from 4 to 30. Examples that may be mentioned of silicone surfactants of formula (IV) include the compounds of formula (IVa)

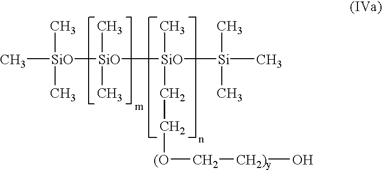

in which m is an integer ranging from 20 to 105, n is an integer ranging from 2 to 10 and y is an integer ranging from 10 to 20,

or the compounds of formula (IVb)

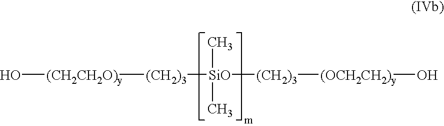

in which m and y are integers ranging from 10 to 20.

Silicone surfactants that may be used in particular are those sold by the company Dow Corning under the names DC 5329 (compound of formula (IVa) in which m=22, n=2, y=12), DC 7439-146 (compound of formula (IVa) in which m=103, n=10, y=12), DC 2-5695 (compound of formula (IVa) in which m=27, n=3, y=12) and Q4-3667 (compound of formula (IIb) in which m=15 and y=13).

(f) diblock copolymers of ethylene oxide and of propylene oxide;

(g) diblock copolymers of styrene and of ethylene oxide, such as, for example, the products sold by the company Goldschmidt under the names SE0418 (PS400/OE1800), SE0720 (PS700/OE2000), SE1010 (PS1000/OE1000), SE1030 (PS1000/OE3000), or the anionic sulphate derivatives of these copolymers, such as SE1030A sold by the company Goldschmidt;

(h) fatty acid esters of sugars and fatty alkyl ethers of sugars, and in particular esters of C₈₋₂₂ fatty acids and of sucrose, maltose, glucose or fructose, or esters of C₁₄₋₂₂ fatty acids and of methylglucose;

(i) (C₁₂₋₃₀)alkenylsuccinates chosen from polyalkoxylated alkenylsuccinates, polyalkoxylated glycose akenylsuccinates and polyalkoxylated methylglucose alkenylsuccinates, such as, for example, PEG hexadecenylsuccinate (18 or 45 EO) and PEG dihexadecenylsuccinate (18 EO);

(j) polyethoxylated acetylenediols such as ethoxylated 2,4,7,9-tetramethyl-5-decyne-4,7-diol (1.3 EO) sold by the company Air Product Chemical under the name Surfinol® 402;

(k) sodium (C₁₂₋₃₀)alkyl ether sulphates, such as sodium lauryl ether sulphate (2.2 EO on average) and sodium (C₁₂₋₃₀)alkyl sulphates, such as sodium lauryl sulphate, sodium tridecyl sulphate and sodium cetylstearyl sulphate (50/50);

(1) quaternary ammonium salts having surfactant properties, corresponding to the following formula:

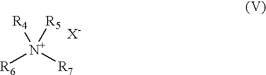

in which

the radicals R₄, R₅, R₆ and R₇ each independently represent a linear or branched aliphatic group or an aromatic group such as an aryl or alkylaryl nucleus, containing from 1 to 30 carbon atoms; the aliphatic groups may comprise hetero atoms such as an oxygen, nitrogen, sulphur or halogen atom, and are chosen, for example, from alkyl, alkoxy, polyoxy(C₂₋₆ alkylene), alkylamide, (C₁₂₋₂₂ alkyl)amido(C₂₋₆ alkyl) , (C₁₂₋₂₂ alkyl)acetate or hydroxyalkyl radicals, containing from 1 to about 30 carbon atoms;

X represents an anion chosen from halide, phosphate, acetate, lactate, (C₂₋₆)alkyl sulphate, (C₂-₆)alkyl sulphonate and (C₂6)alkylaryl sulphonate ions.

The lipophilic active principles may be placed in the form of aqueous suspensions of nanospheres with a mean size ranging from 0.01 to 1 μm, especially according to two known processes.

The first process, known as “solvent nano-precipitation”, is described, for example, in patent application EP-A-0 274 961.

When applied to the preparation of the aqueous suspensions of nanospheres of the present invention, this solvent nanoprecipitation process consists

in dissolving the lipophilic active principle to a concentration ranging from 0.1% to 30% by weight in an organic solvent that is more volatile than water and water-miscible,

in introducing, with moderate stirring, the organic solution of the active principle into an aqueous phase, at least one of these two phases containing at least one dissolved surfactant in a concentration that is greater than or equal to its critical micelle concentration, and then

in evaporating off the organic solvent that is more volatile than water, and also optionally some of the water.

In this process, by virtue of the presence of at least one surfactant, the nanospheres are spontaneously formed by precipitation of the active principle during the mixing of the organic solution of the active principle and of the aqueous phase. The volume of aqueous phase must be sufficient to obtain a satisfactory precipitation of the lipophilic active principle. In practice, it is never less than that of the organic solution.

The aqueous phase/organic phase weight ratio is at least equal to 1 and preferably ranges from 1 to 20.

The size of the nanospheres depends in particular on the nature of the solvent, the concentration of the active principle therein, the organic phase/aqueous phase ratio, and also the nature and amount of the surfactant.

Solvents which may be mentioned that are more volatile than water and water-miscible include ketones such as acetone, C₁₋₆ alcohols such as methanol or isopropanol, tetrahydrofuran, and also mixtures of these solvents.

The second type of process for preparing the aqueous suspensions of nanospheres of the present invention differs from the first process described above mainly by the immiscibility of the organic phase with the aqueous phase. The mixing of the organic solution of the lipophilic active principle and of the aqueous phase will thus give rise to an oil-in-water emulsion.

This “emulsification” process consists in particular

in dissolving the lipophilic active principle to a concentration ranging from 0.1% to 30% by weight in an organic solvent that is more volatile than water and water-immiscible,

in emulsifying the organic solution of the active principle with an aqueous phase, at least one of these two phases containing at least one surfactant at a concentration that is greater than or equal to its critical micelle concentration, and then

in rapidly evaporating off the organic solvent that is more volatile than water, and optionally some of the water.

The organic solvents that are more volatile than water and water-immiscible are chosen, for example, from halogenated hydrocarbons such as dichloromethane, and cyclic hydrocarbons such as cyclohexane and toluene. The aqueous phase/organic phase weight ratio during the emulsification step is between 1.5 and 99.

Contrary to what takes place in the first type of process, the nanospheres do not form spontaneously, and it is generally necessary to refine the pre-emulsion obtained by homogenizing it one or more times in a high-pressure homogenizer (10 to 120 MPa) or by exposing it to ultrasound. The size of the nanospheres obtained will depend directly on the efficacy of this forced emulsification step.

The aqueous suspensions of nanospheres obtained according to one of the two types of process described above may be concentrated by removing a certain amount of the aqueous phase. This removal may take place, for example, by evaporation under vacuum or by ultrafiltration.

Large concentrations of active principle for which the stability of the nanospheres in suspension remains satisfactory will generally be desired, in other words the active principle content of the aqueous suspensions of nanospheres will preferably be adjusted to a value ranging from 0.2% to 50% by weight and preferably from 1% to 20% by weight, relative to the total weight of the aqueous suspension of nanospheres.

The small mean size of the nanospheres is essential for satisfactory bioavailability of the lipophilic active principles, and nanospheres with a mean size ranging from 50 to 500 nm are preferred in particular.

The addition according to the invention of one or more water-dispersible polymers to the aqueous suspensions of nanospheres takes place after these suspensions have been prepared, that is to say after evaporation of the volatile organic phase.

When the aqueous suspensions of nanospheres are concentrated by evaporation or ultrafiltration of a fraction of the aqueous phase, the addition of the particles of water-dispersible polymer preferably precedes the concentration step. However, the water-dispersible polymer may also be added after concentrating the aqueous suspension.

It emerges from the foregoing text that the aqueous suspensions of the present invention contain two types of nanometric particles, namely nanospheres of lipophilic active principle and nanoparticles of water-dispersible polymers. This structure is consequently different from that of the composite nanospheres described in the prior art in which the active principle is found incorporated in a polymer matrix. Such composite nanospheres are described, for example, in the article by Seijo et al., International Journal of Pharmaceutics, 62 (1990), pages 1-7, and in the article by Paul et al., International Journal of Pharmaceutics, 159, (1997), pages 223-232.

According to the present invention, the expression “water-dispersible polymers” means water-insoluble polymers which, when they are dispersed, with moderate to vigorous stirring, in water at a temperature of between 10 and 90° C., spontaneously form colloidal particles with a mean size ranging from 10 to 500 nm.

It is particularly preferred according to the present invention to use colloidal particles of water-dispersible polymer with a mean size ranging from 20 to 400 nm.

The water-dispersible polymers preferably used according to the present invention are synthetic polymers or polymers of natural origin, bearing anionic charges.

As water-dispersible anionic polymers of natural origin which may be used according to the present invention, mention may be made, for example, of anionic derivatives of cellulose, and in particular anionic cellulose esters and ethers such as cellulose acetophthalate, cellulose acetosuccinate, cellulose propionosuccinate, cellulose butyrosuccinate, cellulose acetopropionosuccinate, cellulose acetotrimellitate, cellulose acetopropionotrimellitate, cellulose aceto-butyrotrimellitate and carboxymethylcellulose.

Other anionic water-dispersible polymers of natural origin that may be used are shellac resin, sandarac gum and dammar resins.

Shellac resin is an animal secretion composed mainly of resin and wax and is soluble in certain organic solvents. It should be under-neutralized so as not to become water-soluble.

Sandarac gum is a resin extracted from the bark of trees such as Thuya articulata or Callitris verrucosa. It is composed mainly of acids such as pimeric acid, callitrolic acid and sandaricinic acid. It is insoluble in water but may be dissolved in organic solvents such as ethanol, acetone or ether.

Dammar resins are resins derived from trees of the genera Damara or Shorea and generally contain 62.5% resins (40% soluble and 22.5% insoluble in alcohol) and 23% acids.

The anionic water-dispersible polymers used in the present invention are preferably synthetic anionic polymers and in particular synthetic polymers chosen from polyesters, poly(esteramide), polyurethanes and vinyl copolymers all bearing carboxylic acid and/or sulphonic acid functions.

The anionic polyesters are obtained by polycondensation of aliphatic, cycloaliphatic and/or aromatic dicarboxylic acids and aliphatic, cycloaliphatic and/or aromatic diols or polyols, a certain number of these diacids and diols also bearing a carboxylic acid or sulphonic acid function in free form or in the form of a salt.

Dicarboxylic acids which may be mentioned are succinic acid, glutaric acid, adipic acid, pimelic acid, suberic acid, sebacic acid, terephthalic acid, isophthalic acid or the anhydride thereof.

Aliphatic diols which may be mentioned are ethylene glycol, diethylene glycol, triethylene glycol and tetraethylene glycol, di(hydroxymethyl)cyclohexane, dimethylolpropane and 4,4′-(1-methylpropylidene)-bisphenol.

The polyol monomers are, for example, glycerol, pentaerythritol or sorbitol.

The comonomers which allow anionic groups to be introduced are, for example, dimethylolpropionic acid, trimellitic acid or mellitic anhydride, or a diol or dicarboxylic acid compound also bearing a group SO₃M in which M represents a hydrogen atom or an alkali metal ion, such as sodium 1,5-dihydroxypentane-3-sulphonate or sodium 1,3-dicarboxybenzene-5-sulphonate.

The poly(esteramides) which can be used in the process of the present invention have a structure similar to that of the polyesters described above, but also contain units derived from a diamine such as hexa-methylenediamine, meta- or para-phenylenediamine, or from an amino alcohol such as methanolamine.

According to one preferred embodiment of the invention, the water-dispersible anionic polymer is chosen from aromatic, cycloaliphatic and/or aliphatic polyesters bearing sulphonic acid functions, i.e. copolyesters comprising at least a number of units derived from isophthalic acid, from sulphoaryldicarboxylic acid and from diethylene glycol. Among these, mention may be made most particularly of polyesters comprising units derived from isophthalic acid, from sulphoisophthalic acid, from diethylene glycol and from 1,4-di(hydroxymethyl)cyclohexane, such as those sold under the names AQ®29, AQ®38, AQ®48 Ultra, AQ®55S, AQ®1350, AQ®1045, AQ®1950 and AQ®14000 by the company Eastman Chemical.

These polyesters can also contain units derived from isophthalic acid and from sulphoisopthalic acid, units derived from ethylene glycol, from triethylene glycol and/or from tetraethylene glycol and from terephthalic acid, such as those sold under the names Polycare PS 20, Polycare PS 30 and Polycare PS 32 by the company Rhône-Poulenc.

The proportion of units derived from sulphoisophthalic acid is preferably between 2 and 20% by weight.

The polyurethanes which can be used as water-dispersible anionic polymers are, for example, anionic polyurethane-poly(acrylic acid) copolymers or anionic polyurethane-polyester or poly(ester urethane) copolymers.

The vinyl copolymers which can be used as anionic water-dispersible polymers in particular encompass film-forming polymers commonly used for the preparation of cosmetic compositions, among which mention may be made of:

(i) vinyl acetate/crotonic acid polyethoxylated copolymers, such as the product sold under the name Aristoflex A by the company Hoechst;

(ii) vinyl acetate/crotonic acid copolymers, such as the product sold under the name Luviset CA66 by the company BASF;

(iii) vinyl acetate/crotonic acid/vinyl neo-decanoate terpolymers, such as the product sold under the name Resin 28-29-30 by the company National Starch;

(iv) N-octylacrylamide/methyl methacrylate/hydroxypropyl methacrylate/acrylic acid/tert-butyl-aminoethyl methacrylate copolymers, such as the product sold under the name Amphomer by the company National Starch;

(v) methyl vinyl ether/maleic anhydride alternating copolymers monoesterified with butanol, such as the product sold under the name Gantrez ES 425 by the company GAF;

(vi) acrylic acid/ethyl acrylate/N-tert-butyl-acrylamide terpolymers, such as the product sold under the name Ultrahold® 8 by the company BASF;

(vii) the polymers corresponding to the following general formula

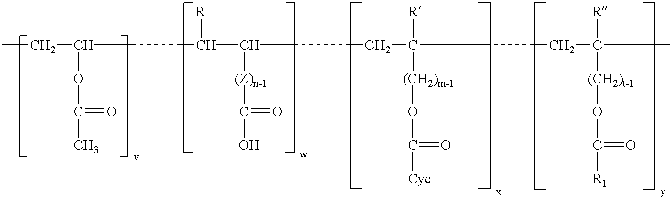

in which

R, R′ and R″, which may be identical or different, represent a hydrogen atom or a methyl radical,

m, n and t are equal to 1 or 2,

R₁ represents a linear or branched, saturated or unsaturated C₂-C₂₁ alkyl radical,

z represents a divalent radical chosen from the residues:

—CH₂—, —CH₂—O—CH₂— and CH₂—O—(CH₂)₂—,

Cyc represents a radical chosen from:

(a) a radical of formula

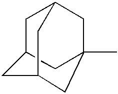

(b) a radical of formula

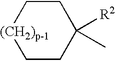

in which R₂ represents a hydrogen atom or a methyl radical and p is equal to 1 or 2,

(c) a radical of formula

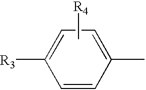

in which R₃ represents a hydrogen atom, a methyl, ethyl, tert-butyl, ethoxy, butoxy or dodecyloxy radical and R₄ represents a hydrogen atom, a C₁-C₄ alkyl radical or a C₁-C₄ alkoxy radical, and

(d) a radical of formula

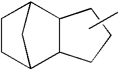

v is chosen such that the corresponding units represent from 10 to 91% by weight, preferably from 36 to 84% by weight, of the total polymer,

w is chosen such that the corresponding units represent from 3 to 20% by weight, preferably from 6 to 12% by weight, of the total polymer,

x is chosen such that the corresponding units represent from 4 to 60% by weight, preferably from 6 to 40% by weight, of the total polymer, and

y is chosen such that the corresponding units represent from 0 to 40% by weight, preferably from 4 to 30% by weight, of the total polymer the sum of v+w+x+y being equal to 100%.

Among these polymers, those which may be mentioned in particular are the vinyl acetate/vinyl 4-tert-butylbenzoate/crotonic acid (65/25/10) copolymer neutralized to 50-60% with lysine, and the vinyl acetate/crotonic acid/vinyl 4-tert-butylbenzoate (65/10/25) copolymer neutralized to 60% with lysine.

The weight-average molar mass of the water-dispersible anionic polymers used in the present invention for the stabilization of suspensions of nanospheres of lipophilic principle generally ranges from 1000 to 5000000 and preferably from 5000 to 500000.

As mentioned above, the anionic water-dispersible polymers described above must be water-insoluble. However, the presence of the anionic charges increases their polarity and promotes their dissolution in water. It is consequently essential to limit the content of charge in the polymers.

This upper charge-content limit which should not be exceeded in order for the polymer to remain insoluble depends

on the chemical nature of the polymer, i.e. the hydrophobic nature of the units of which it is composed,

on the molar mass of the polymer, a polymer of low molar mass generally being more soluble in water than a polymer of high mass, or

on the nature of the agent for neutralizing the acid functions.

It is possible to modify this charge content by varying the content of comonomers introducing carboxylic acid or sulphonic acid functions or by varying the degree of neutralization of the weak acid groups (carboxylic acid groups).

The partial neutralization (under-neutralization) of the weak acid functions can be carried out by adding a non-volatile monobasic agent, such as an inorganic base, for instance sodium hydroxide or potassium hydroxide, or an amino alcohol taken from the group consisting of 2-amino-2-methyl-l-propanol (AMP), triethanolamine (TEA), triisopropanolamine (TIPA), monoethanolamine, diethanolamine, tris[(2-hydroxy)-1-propyl]amine, 2-amino-2-methyl-1,3-propanediol (AMPD) and 2-amino-2-hydroxymethyl-1,3-propanediol.

About 20 to 80% of the ionizable groups can thus be neutralized in order to stabilize the aqueous dispersion without dissolving the polymer.

In the present application, the expression “amount that is sufficient to stabilize the nanospheres against recrystallization of the active principle” means an amount of water-dispersible polymer that makes it possible to obtain aqueous suspensions showing no sign of visible change by microscope (cross-polarization, phase contrast) at a magnification suited to the size of the particles, and in particular no sign of recrystallization of the active principle after storage for at least 7 days at a temperature of between 4° C. and 45° C.

The amount of water-dispersible polymer required to obtain a satisfactory stabilization of the aqueous suspensions of nanospheres of lipophilic active principle depends on many parameters, such as the content of charge and the chemical nature of the water-dispersible polymer, the chemical nature and the concentration of active principle, or the nature and concentration of the surfactant used.

The Applicant has found that a water-dispersible polymer/lipophilic active principle weight ratio ranging from 1/100 to 1/1 generally gives satisfactory results, that is to say aqueous suspensions showing no signs of crystallization after 7 days and even after 2 months or more of storage at a temperature of between 4 and 45° C.

This water-dispersible polymer/lipophilic active principle weight ratio preferably ranges from 1/50 to 1/2.

A subject of the present invention is also topical-application compositions containing an aqueous suspension of nanospheres of lipophilic active principle stabilized with water-dispersible polymers.

These topical-application compositions contain from 0.1% to 40% by weight and preferably from 1% to 30% by weight of aqueous suspension of nanoparticles of active principle, in a physiologically acceptable medium.

These compositions may be, for example, in the form of lotions, gels, suspensions, emulsions such as W/O or O/W emulsions, W/O/W or O/W/O multiple emulsions, or nanoemulsions.

They may contain additives or adjuvants usually used in cosmetics, such as antioxidants, essential oils, moisturizers, vitamins, essential fatty acids, sphingo-lipids, self-tanning agents, free-radical scavengers, sunscreens, fragrances, preserving agents, colorants, antifoams, sequestering agents, pH regulators, hydrophilic thickeners such as polysaccharides (xanthan gum), carbomers (carboxyvinyl polymers), or partially neutralized and highly crosslinked polyacrylamido-methylpropanesulphonic acid.

Needless to say, a person skilled in the art will take care to select the optional additional compounds such that the advantageous properties of the composition according to the invention, and especially the stability of the nanospheres, are not, or are virtually not, adversely affected by the addition envisaged.

The composition according to the invention may be used in many cosmetic or dermatological applications in which the presence of lipophilic active agents is useful, especially to treat, care for and/or make up facial and/or body skin, mucous membranes (lips), the scalp and/or keratin fibres (hair or eyelashes).

Thus, the composition of the invention may be used as a care and/or hygiene product or as an antisun product for the face, the hands or the body. It may also constitute a make-up product for keratin fibres, the skin, the lips and/or the nails.

The composition according to the invention may also be used as a rinse-out or leave-in hair product, in particular for washing, caring for, conditioning or maintaining the hairstyle or for shaping keratin fibres such as the hair.

Thus, a subject of the present invention is also the cosmetic use of the composition according to the invention to treat, care for and/or make up facial and/or body skin, mucous membranes (lips), the scalp and/or keratin fibres.

Another subject of the invention is a cosmetic treatment process for human keratin materials such as the skin, including the scalp, the hair, the eyelashes, the eyebrows, the nails or mucous membranes, especially the lips, characterized in that a cosmetic composition as defined above is applied to the keratin materials, according to the usual technique for using this composition. For example, application of creams, gels, sera, lotions or milks to the skin, the scalp and/or mucous membranes. This type of treatment depends on the active agent(s) present in the composition.

The present invention will be illustrated with the aid of the examples which follow.

### EXAMPLE 1

### Stability of Aqueous Suspensions of N-cholesteryloxycarbonyl-4-aminophenol

An aqueous suspension of nanoparticles of N-cholesteryloxycarbonyl-4-aminophenol stabilized with a water-dispersible polymer and also a comparative suspension free of water-dispersible polymer are prepared according to the solvent nanoprecipitation process described above.

These suspensions are stored for 2 months at a temperature of 45° C., after which period the degree of crystallization of the active principle is evaluated by polarization microscopy and by phase-contrast microscopy. The composition of the suspensions and the results obtained are collated in the table below.

| | Suspension A | |
|---|---|---|
| | (according to the | Comparative |
| | invention) | suspension |
| N-cholesteryloxy- | 3% by weight | 3% by weight |
| carbonyl-4-amino- | | |
| phenol | | |
| Soybean lecithin | 0.5% by weight | 0.5% by weight |
| Aqueous suspension | 20% by weight | — |
| of AQ ® 38S*) at 6% | | |
| by weight | | |
| Distilled water | qs 100% by weight | qs 100% by weight |
| Result of the | Absence of | Extensive |
| microscopic | crystallization | crystallization |
| examination | after storage for | after storage for |
| | 2 months at 45° C. | 2 months at 45° C. |

*) Copolyester of isophthalic acid, of sulphoisophthalic acid, of dimethylolcyclohexane and of diethylene glycol, sold by the company Eastman Chemical

These results show that the addition, to an aqueous suspension of nanoparticles of N-cholesteryloxycarbonyl-4-aminophenol, of only 1.2% by weight of a water-dispersible polymer according to the present invention allows the suspension to be stabilized perfectly for at least 2 months.

### EXAMPLE 2

An aqueous suspension containing 5% by weight of nanoparticles of N-cholesteryloxycarbonyl-4-aminophenol, 3% by weight of soybean lecithin and 1.2% by weight of water-dispersible polymer according to the invention (AQ®38S) is prepared according to the emulsification 20 process described above. After storage of the aqueous suspension for 2 months at a temperature of 45° C., the examination by cross-polarization microscopy and by phase-contrast microscopy reveals the absence of crystallized particles of active principle. The nanospheres of N-cholesteryloxycarbonyl-4-aminophenol in the suspension have a mean size, measured by laser granulometry (BI90 Plus from Brookhaven (QLS method)) of 131 nm, which is identical to that measured for the suspension before storage.

### EXAMPLE 3

An aqueous suspension containing 9.3% by weight of nanoparticles of N-cholesteryloxycarbonyl-4-amino-phenol, 4.8% by weight of soybean lecithin and 1.2% by weight of water-dispersible polymer according to the invention (AQ®38S) is prepared according to the emulsification process described above. After storage of the aqueous suspension for 2 months at a temperature of 45° C., the examination by cross-polarization microscopy and by phase-contrast microscopy reveals the absence of crystallized particles of active principle. The nanospheres of N-cholesteryloxycarbonyl-4-aminophenol in the suspension have a mean size, measured by laser granulometry (BI90 Plus from Brookhaven (QLS method)) of 112 nm, which is identical to that measured for the suspension before storage.

### EXAMPLE 4

An aqueous suspension containing 1.5% by weight of nanoparticles of ursolic acid with a mean size, measured by laser granulometry (BI90 Plus from Brookhaven (QLS method)), of 112 nm, 0.8% of soybean lecithin and 0.8% by weight of water-dispersible polymer according to the invention (AQ®38S) is prepared according to the solvent nanoprecipitation process described above. The suspension, stored at a temperature of 45° C., is perfectly stable for at least 2 months, whereas the same suspension free of water-dispersible polymer is the site of extensive crystallization within less than 7 days of storage at 45° C.

1. Stable colloidal suspension consisting essentially:
of a continuous aqueous phase,
of nanospheres of lipophilic active principle with a mean particle size ranging from 0.01 to 1 μm,
of at least one surfactant, and
of colloidal particles, with a mean size ranging from 10 to 500 nm, of at least one water-dispersible polymer.

2. Stable colloidal suspension according to claim 1, characterized in that the lipophilic active principle is chosen from sterols of plant and animal origin and partially hydrogenated esters and/or derivatives thereof, dehydroepiandrosterone (DHEA) and chemical and biological precursors and derivatives thereof, pentacyclic triterpene acids, hydroxystilbenes, isoflavonoids, and aminophenol derivatives of formula 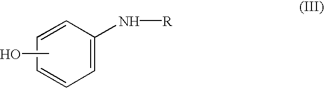
in which R is a radical corresponding to one of the formulae (i), (ii) and (iii) below
—CO—NR₁R₂ (i)
—CO—O—R₃ (ii)
—SO₂—R₃ (iii)
in which
R₁ represents a hydrogen atom or an optionally hydroxylated, saturated or unsaturated, linear or branched C₁₋₆ alkyl radical,
R₂ represents a hydrogen atom or a radical chosen from optionally hydroxylated, saturated or unsaturated, linear, cyclic or branched C₁₂ to C₃₀ alkyl radicals, and
R₃ represents a radical chosen from saturated or unsaturated, linear, branched or cyclic C₁₂ to C₃₀ alkyl radicals, including fused polycyclic radicals, that are optionally hydroxylated.

3. Stable colloidal suspension according to claim 2, characterized in that the active principle is chosen from dehydroepiandrosterone (DHEA), DHEA sulphate, 7α-hydroxy-DHEA, 7-keto-DHEA, prednisolone, prednisone, progesterone, pregnenolone, testosterone, diosgenin, hecogenin, ursolic acid, oleanolic acid, resveratrol (=3,5,4′-trihydroxystilbene) and N-cholesteryl-oxycarbonyl-4-aminophenol, and isoflavonoids whose solubility in water at room temperature (25° C.) is less than 0.01%.

4. Stable colloidal suspension according to any one of the preceding claims, characterized in that the surfactant(s) is(are) chosen from nonionic, anionic, cationic and zwitterionic surfactants, and mixtures thereof.

5. Stable colloidal suspension according to any one of the preceding claims, characterized in that the surfactant(s) is(are) chosen from
(a) hydrogenated or non-hydrogenated natural or synthetic phospholipids, optionally enriched with phosphatidylcholine,
(b) polyethoxylated sterols,
(c) surfactants chosen from fatty esters of glycerol, fatty esters of sorbitan, polyethoxylated fatty esters of sorbitan, polyethoxylated fatty alcohols and polyethoxylated fatty acids, the fatty chains of these molecules being saturated, and linear or branched;
(d) poly(vinyl alcohol), polyvinylpyrrolidone and copolymers thereof,
(e) polyethoxylated, and optionally poly-propoxylated, polysiloxanes (CTFA name: dimethicone copolyols),
(f) diblock copolymers of ethylene oxide and of propylene oxide,
(g) diblock copolymers of styrene and of ethylene oxide and anionic sulphate derivatives thereof,
(h) fatty acid esters of sugars and fatty alkyl ethers of sugars, and in particular esters of C₈₋₂₂ fatty acids and of sucrose, maltose, glucose or fructose, or esters of C₁₄₋₂₂ fatty acids and of methylglucose, and mixtures thereof;
(i) alkenylsuccinates chosen from polyalkoxylated alkenylsuccinates, polyalkoxylated glucose akenylsuccinates and polyalkoxylated methylglucose alkenylsuccinates,
(j) polyethoxylated acetylenediols,
(k) sodium (C₁₂₋₃₀)alkyl ether sulphates and sodium (C₁₂₋₃₀)alkyl sulphates,
(l) quaternary ammonium salts having surfactant properties, corresponding to the following formula: 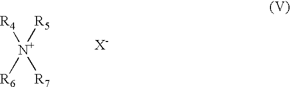
in which the radicals R₄, R₅, R₆ and R₇ each independently represent a linear or branched aliphatic group or an aromatic group such as an aryl or alkylaryl nucleus, containing from 1 to 30 carbon atoms, and X is chosen from halide, phosphate, acetate, lactate, (C₂₋₆)alkyl sulphate, (C₂₋₆)alkyl sulphonate or (C₂₋₆)alkylaryl sulphonate ions.

6. Stable colloidal suspension according to any one of the preceding claims, characterized in that the surfactant(s) is(are) present in a concentration ranging from 0.02% to 25% relative to the total weight of the suspension.

7. Stable colloidal suspension according to any one of the preceding claims, characterized in that the water-dispersible polymers are synthetic polymers or polymers of natural origin, bearing anionic charges.

8. Stable colloidal suspension according to claim 7, characterized in that the water-dispersible polymers of natural origin are chosen from anionic cellulose derivatives such as anionic cellulose esters and ethers, shellac resin, sandarac gum and dammar resins.

9. Stable colloidal suspension according to claim 7, characterized in that the synthetic water-dispersible polymers are chosen from polyesters, poly(esteramide), polyurethanes and vinyl copolymers, bearing carboxylic acid and/or sulphonic acid functions.

10. Stable colloidal suspension according to claim 9, characterized in that the water-dispersible polymers are chosen from aromatic, cycloaliphatic and/or aliphatic polyesters bearing sulphonic acid functions.

11. Stable colloidal suspension according to claim 10, characterized in that the water-dispersible polymers are chosen from copolyesters consisting of units derived from isophthalic acid, from sulphoisophthalic acid, from diethylene glycol and from 1,4-di(hydroxymethyl)cyclohexane.

12. Stable colloidal suspension according to claim 10, characterized in that the water-dispersible polymers are chosen from copolyesters consisting of units derived from isophthalic acid, from sulphoisophthalic acid, from ethylene glycol and from terephthalic acid.

13. Stable colloidal suspension according to one of claims 10 to 12, characterized in that the fraction of units derived from sulphoisophthalic acid in the polymer ranges from 2% to 20% by weight.

14. Stable colloidal suspension according to any one of the preceding claims, characterized in that the mean size of the colloidal particles of water-dispersible polymer ranges from 20 to 400 nm.

15. Stable colloidal suspension according to any one of the preceding claims, characterized in that the water-dispersible polymer/nanospheres of lipophilic active principle weight ratio ranges from 1/100 to 1/1 and preferably from 1/50 to 1/2.

16. Topical-application composition containing, in a physiologically acceptable medium, a stable colloidal suspension according to any one of the preceding claims.

17. Topical-application composition according to claim 16, characterized in that it contains from 0.1% to 40% by weight, preferably from 1% to 30% by weight of stable colloidal suspension according to any one of claims 1 to 14.

18. Use of water-dispersible polymers in the form of colloidal particles with a mean size ranging from 10 to 500 nm to stabilize aqueous suspensions of nanospheres of lipophilic active principle, with a mean particle size ranging from 0.01 to 1 μm, against the recrystallization of the active principle.

19. Use according to claim 18, characterized in that the lipophilic active principle is chosen from sterols of plant and animal origin and partially hydrogenated esters and/or derivatives thereof, dehydroepiandrosterone (DHEA) and chemical and biological precursors and derivatives thereof, pentacyclic triterpene acids, hydroxystilbenes, isoflavonoids, and aminophenol derivatives of formula 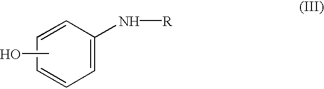
in which R is a radical corresponding to one of the formulae (i), (ii) and (iii) below
—CO—NR₁R₂ (i)
—CO—O—R₃ (ii)
—SO₂—R₃ (iii)
in which
R₁ represents a hydrogen atom or an optionally hydroxylated, saturated or unsaturated, linear or branched C₁₋₆ alkyl radical,
R₂ represents a hydrogen atom or a radical chosen from optionally hydroxylated, saturated or unsaturated, linear, cyclic or branched C₁₂ to C₃₀ alkyl radicals, and
R₃ represents a radical chosen from saturated or unsaturated, linear, branched or cyclic C₁₂ to C₃₀ alkyl radicals, including fused polycyclic radicals, that are optionally hydroxylated.

20. Use according to claim 19, characterized in that the active principle is chosen from dehydroepiandrosterone (DHEA), DHEA sulphate, 7α-hydroxy-DHEA, 7-keto-DHEA, prednisolone, prednisone, progesterone, pregnenolone, testosterone, diosgenin, hecogenin, ursolic acid, oleanolic acid, resveratrol (=3,5,4′-trihydroxystilbene) and N-cholesteryloxycarbonyl-4-aminophenol, and isoflavonoids whose solubility in water at room temperature (25° C.) is less than 0.01%.

21. Use according to any one of claims 18 to 20, characterized in that the water-dispersible polymers are synthetic polymers or polymers of natural origin, bearing anionic charges.

22. Use according to claim 21, characterized in that the water-dispersible polymers of natural origin are chosen from anionic cellulose derivatives such as anionic cellulose esters and ethers, shellac resin, sandarac gum and dammar resins.

23. Use according to claim 21, characterized in that the synthetic water-dispersible polymers are chosen from polyesters, poly(esteramide), polyurethanes and vinyl copolymers, bearing carboxylic acid and/or sulphonic acid functions.

24. Use according to claim 23, characterized in that the water-dispersible polymers are chosen from aromatic, cycloaliphatic and/or aliphatic polyesters bearing sulphonic acid functions.

25. Use according to claim 24, characterized in that the water-dispersible polymers are chosen from copolyesters consisting of units derived from isophthalic acid, from sulphoisophthalic acid, from diethylene glycol and from 1,4-di(hydroxymethyl)cyclohexane.

26. Use according to claim 25, characterized in that the water-dispersible polymers are chosen from copolyesters consisting of units derived from isophthalic acid, from sulphoisophthalic acid, from ethylene glycol and from terephthalic acid.

27. Use according to one of claims 24 to 26, characterized in that the fraction of units derived from sulphoisophthalic acid in the polymer ranges from 2% to 20% by weight.

28. Use according to any one of claims 18 to 27, characterized in that the mean size of the colloidal particles of water-dispersible polymer ranges from 20 to 400 nm.

29. Use according to any one of claims 18 to 28, characterized in that the water-dispersible polymer/nanospheres of lipophilic active principle weight ratio ranges from 1/100 to 1/1 and preferably from 1/50 to 1/2.

30. Cosmetic use of the composition according to either of claims 16 and 17, to treat, care for and/or make up facial and/or body skin, mucous membranes, the scalp and/or keratin fibres.

31. Cosmetic treatment process for human keratin materials such as the skin, including the scalp, the hair, the eyelashes, the eyebrows, the nails or mucous membranes, especially the lips, characterized in that a composition according to either of claims 16 and 17 is applied to the keratin materials.
